(19) United States
(12) Patent Application Publication
(10) Pub. No.: US 2005/0079237 A1
(43) Pub. Date: Apr. 14, 2005

(54) Methods for preparing dry-heating seeds of Canavalia ensiformis and compositions comprising the same for immunomodulation

(76) Inventor: Yang Chi Chiang, Taichung (TW)

Correspondence Address: HARNESS, DICKEY & PIERCE, P.L.C., P.O. Box 8910, Reston, VA, 20195, US

(21) Appl. No.: 10/926,323

(22) Filed: Aug. 26, 2004

Related U.S. Application Data
(60) Provisional application No. 60/497,905, filed on Aug. 27, 2003.

(51) Int. Cl. A61K 35/78

(52) U.S. Cl. 424/776

(57) ABSTRACT

The invention provides methods for preparing dry-heating seeds of Canavalia ensiformis (C. ensiformis) and compositions for immunomodulation comprising the same. The invention also provides methods for the treatment of diseases caused or affected by malfunction of the immune system.

### FIELD OF THE INVENTION

The invention relates to methods for preparing dry-heating seeds of Canavalia ensiformis (C. ensiformis) and to compositions comprising the same for immunomodulation. The invention also relates to methods for the treatment of diseases caused or affected by malfunction of the immune system.

### DESCRIPTION OF PRIOR ART

Canavalia ensiformis (jack bean) is a good source of protein (23% to 34%), carbohydrate (55%) and minerals Na, K, Ca, Mg, P, Fe, Zn, Cu, Mn and Ni. C. ensiformis is a tropical legume, which is commonly used in animal feeding. Planting of C. ensiformis was recorded in local Taiwan archives as early as the Ming Dynasty (AD 1368-1644). In India, seeds of C. ensiformis are a traditional food of the aborigines. However, certain antinutritional and toxic factors, including lectins, hemagglutinins, cyanogen glucosides, oligosaccharides and trypsin inhibitors, are present in seeds of C. ensiformis and have hindered its use in more civilized society.

Lectin concanavalin A (Con A) constitutes up to 15% of the protein in the cotyledons of seeds of C. ensiformis (Carrington DM, et al, 1985. Nature 313: 64-67). Although harmful to animals, Con A is a well-known polyclonal mitogen that has been reported to induce human and mouse T-cell mitosis (Barral-Netto M, et al, 1992. Immunol Invest 21: 297-303; Rodriguez D, et al, 1992. Braz J Med Biol Res 25: 823-826). Con A has been demonstrated to activate T cells which produce cytokines and cytokine receptor in vitro. The increased cytokine activity induced the proliferation of the selected clones and the production of memory T cells.

Since seeds of C. ensiformis have been consumed by different species for a long time (Marfo EK, et al, 1990. Gen Pharmacol 21: 753-757; Mendez A, et al, 1998. Poult Sci 77: 282-289), the use of C. ensiformis is widely accepted as a safe source of nutrition.

### BRIEF DESCRIPTION OF THE INVENTION

The present invention provides a method of preparing dry-heating seeds of Canavalia ensiformis comprising roasting the seeds of Canavalia ensiformis at more than 170-250° C. for 0.5-150 minutes.

The present invention also provides a composition for use in immunomodulation comprising dry-heating seeds of Canavalia ensiformis wherein the seeds are roasted at more than 170-250° C. for 0.5-150 minutes.

The present invention further provides a method for the treatment of diseases caused or affected by malfunction of the immune system.

### BRIEF DESCRIPTION OF THE DRAWINGS

FIG. 1. The ratio of cytokines in mice given 50% seeds of C. ensiformis roasted at 180° C. (group A1) compared with those of the control group (group D) from months 1 to 6 (P<0.05).

FIG. 2. The ratio of cytokines in mice given 75% seeds of C. ensiformis roasted at 180° C. (group A2) compared with those of the control group (group D) from months 1 to 6 (P<0.05).

FIG. 3. The ratio of cytokines in mice given 50% seeds of C. ensiformis roasted at 190° C. (group B1) compared with those of the control group (group D) from months 1 to 6(P<0.05).

FIG. 4. The ratio of cytokines in mice given 75% seeds of C. ensiformis roasted at 190° C. (group B2) compared with those of the control group (group D) from months 1 to 6(P<0.05).

FIG. 5. The ratio of cytokines in mice given 50% seeds of C. ensiformis roasted at 200° C. (group C1) compared with those of the control group (group D) from months 1 to 6 (P<0.05).

FIG. 6. The ratio of cytokines in mice given 75% seeds of C. ensiformis roasted at 200° C. (group C2) compared with those of the control group (group D) from months 1 to 6 (P<0.05).

### DETAILED DESCRIPTION OF THE INVENTION

The present invention provides a method of preparing dry-heating seeds of Canavalia ensiformis comprising roasting the seeds of Canavalia ensiformis at more than 170-250° C. for 0.5-150 minutes.

In the preparation of the invention, the seeds are roasted at more than 170-250° C. for 0.5-150 minutes, preferably at more than 170-220° C. for 1.5-100 minutes, more preferably at 180-200° C. for 5-50 minutes, and the most preferably at 180° C. for 25 minutes, at 190° C. for 15 minutes or at 200° C. for 5 minutes.

In the following examples, it demonstrated that the treated seeds of Canavalia ensiformis according to the preparation of the present invention.

Accordingly, the present invention further provides a composition for use in immunomodulation comprising dry-heating seeds of Canavalia ensiformis wherein the seeds are roasted at more than 170-250° C. for 0.5-150 minutes.

In the composition of the invention, the concentration of the seeds ranges from 1 to 99, preferably 10 to 90, more preferably 30 to 80 or 40 to 75 and the most preferably 50 to 75 weight percent.

The pharmaceutically, veterinarily or dietetically acceptable excipient may be a solvent (such as water), dispersion medium, coating, isotonic or absorption delaying agent, sweetener or the like. These include any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and adsorbents that may be needed in order to prepare a particular dosage form. The use of such media and agents for pharmaceutically active substances is well known in the art. Except insofar as any conventional media or agent is incompatible with the active ingredient, its use in the present compositions is contemplated.

The present composition can be provided in any convenient form and for administration by any route such as orally, topically or parenterally. It can be provided as a dietary supplement or any pharmaceutical dosage form. It can be formulated into a food or drink, and provided, for example, as a snack bar; a cereal, a drink, a gum, or in any other easily ingested form. It can also be provided as a cream or lotion for topical application.

The term “Immunomodulation” used in the invention is a developing segment of immunopharmacology. Immunomodulator compounds and compositions, as the name implies, are useful for modulating or regulating immunological functions in animals. Immunomodulators may be immunostimulants for building up immunities to, or initiate healing from, certain diseases and disorders. Conversely, immunomodulators may be immunoinhibitors or immunosuppressors for preventing undesirable immune reactions of the body to foreign materials, or to prevent or ameliorate autoimmune reactions or diseases.

Immunomodulators have been found to be useful for treating systemic autoimmune diseases, such as lupus erythematosus and diabetes, as well as immunodeficiency diseases. Further, immunomodulators may be useful for immunotherapy of cancer or to prevent rejections of foreign organs or other tissues in transplants, e.g., kidney, heart, or bone marrow.

Usually, an infectious disease is a disease which arises when a malfunction of the immune system (a self-defense system for protecting the living body (host) from infections by exogenous pathogens, such as viruses, bacteria, parasites and fungi) occurs, so that the exogenous pathogens cannot be excluded from the living body. On the other hand, an autoimmune disease is a disease in which the immune system malfunctions to attack the “self” (which should not be attacked but should be defended by the immune system). Two types of autoimmune diseases, namely the autoimmune diseases specific to an organ or tissue and the non-specific systemic autoimmune diseases, are known in the art. A wide variety of autoimmune diseases are known to result from immunomodulation disorders, and examples of such autoimmune diseases include systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis and Graves' orbitopathy.

Accordingly, the present invention further provides a method for the treatment of immune diseases caused by malfunction of the immune system comprises administering the concentration of 1 to 99 weight percent of dry-heating seeds of Canavalia ensiformis wherein the seeds are roasted at 170-250° C. for 0.5-60 minutes, to a human or animal in need of such treatment.

In the treatment method of the invention, the immune diseases are infections by exogenous pathogens (such as viruses, bacteria, parasites or fungi), autoimmune diseases (such as systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis or Graves' orbitopathy). The present invention can be applied for cancer immunotherapy.

One trained in the art can readily formulate the present composition into any of these convenient forms for oral or topical administration.

### EXAMPLES

### Example 1

### Materials and Methods

Mice and Serum Samples

One hundred and twenty female Balb/c mice, purchased from the National Cheng Kung University (Tainan, Taiwan) at four weeks of age and weighing between 14 and 16 gms, were used in this animal study. The mice were randomly divided into seven groups. Canavalia ensiformis seeds were roasted at 180° C. for 25 min, 190° C. for 15 min and 200° C. for 5 min, and a basal diet for mice was prepared to which either 50%, or 75% of dried-ground Canavalia ensiformis seeds were added respectively. Mice in three groups were fed a 50% diet (180° C.: group A1; 190° C.: group B1; 200° C.: group C1) respectively, and the mice in the other three groups were fed with a 75% diet (180° C.: group A2; 190° C.: group B2; 200° C.: group C2) respectively. Another group was fed with a diet without Canavalia ensiformis seeds and served as the normal control (group D). There were fifteen mice in each group. A minimum of two mice were sacrificed at the end of each month for six months after the regimen had commenced. Sera were separated from the blood samples of sacrificed mice by centrifugation at 3,500×g for 15 min, and aliquoted and stored at −70° C. until required for cytokine assay. The brain, heart, liver, spleen and kidneys were sectioned for hematoxylin-eosin (H&E) staining.

Hematoxylin-Eosin (H&E) Staining

All specimens were examined by routine fixation with H&E staining. All brains, hearts livers, spleens and kidneys were fixed immediately in a 10% formalin solution overnight at 4° C. and embedded in paraffin. Serial sagittal sections 5 μm thick were cut, and stained with H&E for histological study.

Cytokines Determinations by ELISA

Cytokines (IL-2, IL-4, IL-10 and IFN-γ) were assayed using commercial ELISA kits (Pharmingen, San Diego, Calif., USA) according to the manufacturer's instructions as described before (Lin et al., 2004). Using 96-well plates, 100μl of a 1:250 diluted anti-mouse capture antibodies were added and incubated overnight at 25° C. Volumes of 100 μl of sera or standard were added in duplicate and plates were incubated at room temperature for two hours. After incubation, plates were washed five times with ELISA washing solution. Then, 100 μl of working detector (biotinylated anti-mouse monoclonal antibody conjugated to horseradish peroxidase at 1:250 dilution in assay diluents) were added. Plates were incubated at 25° C. for one hour. After incubation, the wells were washed seven times with ELISA washing solution using an ELISA washer (BIO-RAD, Hercules, Calif., USA). Substrate solution (Tetramethylbenzidine) was added and plates were incubated at 37° C. After incubation for 15 min, the reaction was terminated by the stop solution (2N H₂SO₄) and the absorbance was read at 450 nm (Spectrophotometer Model 550, BIO-RAD). For every test, a standard curve was also derived for IL-2, IL-4, IL-10 and IFN-γ. The range of the standard curve was extended as 0.0000 pg ˜1000 pg for IL-2, 0.0000 pg ˜1000 pg for IL-4, 0.0000 pg ˜4000 pg for IL-10, and 0.0000 pg ˜8000 pg for IFN-γ.

Statistical Analyses

The Kruskal-Wallis test was used to test the differences on weight among different groups and P values <0.05 were taken as the significant. The Wilcoxson rank sum test was used to test the weight difference and cytokine ratio between each two different groups and P values <0.05 were taken as the significant.

Results

At the end of each month during the study period, at least two mice from each group were sacrificed. The weights (Table 1) and physiological characteristics of the mice were recorded for each group. The results indicate no weight difference among different groups (P>0.05). Furthermore, there was no pathological change in the brain, the heart, the liver, the spleen and the kidneys of the mice given Canavalia ensiformis seeds (Histology data not shown).

TABLE 1
The mean weight (gms) ± standard deviation of mice fed with 50% (group
A1, B1, C1), 75% (group A2, B2, C2) or 0% (group D) Canavalia ensiformis seed
roasted at different temperature (P > 0.05). Twenty mice were included in each group.

| | Mean weight (gms) ± standard deviation | | | | | | |
|---|---|---|---|---|---|---|---|
| | 180° C. | 180° C. | 190° C. | 190° C. | 200° C. | 200° C. | control |
| Month | Group A1 | Group A2 | Group B1 | Group B2 | Group C1 | Group C2 | Group D |
| 1 | 29.00 ± 1.00 | 32.50 ± 0.50 | 28.50 ± 2.50 | 29.00 ± 1.00 | 31.25 ± 1.75 | 30.00 ± 2.00 | 33.00 ± 0.00 |
| 2 | 29.00 ± 1.00 | 32.50 ± 0.50 | 28.50 ± 2.50 | 29.00 ± 1.00 | 31.25 ± 1.75 | 33.00 ± 5.00 | 32.00 ± 0.00 |
| 3 | 30.50 ± 0.50 | 29.50 ± 0.50 | 30.00 ± 2.00 | 27.00 ± 1.00 | 35.00 ± 1.00 | 30.00 ± 2.00 | 29.65 ± 0.55 |
| 4 | 29.65 ± 0.55 | 27.30 ± 1.10 | 30.10 ± 1.50 | 30.15 ± 1.25 | 27.15 ± 8.55 | 29.30 ± 1.80 | 30.70 ± 0.70 |
| 5 | 35.50 ± 3.10 | 35.85 ± 1.05 | 37.10 ± 2.60 | 37.55 ± 1.95 | 35.75 ± 2.55 | 35.50 ± 1.40 | 34.70 ± 1.00 |
| 6 | 35.32 ± 0.84 | 31.32 ± 2.28 | 27.54 ± 1.60 | 31.30 ± 2.65 | 33.48 ± 4.39 | 34.14 ± 2.95 | 37.80 ± 1.38 |

No mice in any group died during the experimental period. The serum samples from each mouse were collected separately for IL-2, IL-4, IL-10 and IFN-γELISA assays.

Standard curves for the cytokines were also derived and covered a wide range. The cytokine concentration for the control group ranged from 0.3461 to 0.8527 pg/ml for IL-2, 0.0114 to 0.7040 pg/ml for IL-4, 0.0105 to 0.1934 pg/ml for IL-10, and 0.1646 to 2.6615 pg/ml for IFN-γ. The mean cytokine concentrations for mice fed with 50% (groups A1, B1, C1), 75% (groups A2, B2, C2), or 0% (group D) Canavalia ensiformis seed roasted at different temperature was identified. Compared with the control group, the cytokine concentrations for the mice fed with the diet containing Canavalia ensiformis seed show greater variation. When the mice fed with a diet with Canavalia ensiformis seed roasted at 180° C. (group A1), IL-2 varied from 0.3368 to 13.7760 pg/ml, IL-4 from 0.0155 to 2.420 pg/ml, IL-10 from 0.0110 to 0.9028 pg/ml and IFN-γfrom 0.1425 to 25.5032 pg/ml; at 190° C. (group B1), IL-2 varied from 0.3554 to 4.9011 pg/ml, IL-4 from 0.0138 to 5.9409 pg/ml, IL-10 from 0.0148 to 0.7533 pg/ml and IFN-γfrom 0.1633 to 10.7892 pg/ml; and at 200° C. (group C1), IL-2 varied from 0.4390 to 12.4852 pg/ml, IL-4 from 0.0150 to 355.0835 pg/ml, IL-10 from 0.0141 to 8.1083 pg/ml and IFN-γfrom 0.1758 to 38.5015 pg/ml. When the mice fed with the diet with 75% Canavalia ensiformis seed roasted at 180° C. (group A2), IL-2 varied from 0.4628 to 5.7260 pg/ml, IL-4 from 0.0153 to 2.2728 pg/ml, IL-10 from 0.0110 to 5.9058 pg/ml and IFN-γfrom 0.1757 to 3.5817 pg/ml; at 190° C. (group B2), IL-2 varied from 0.4067 to 12.2182 pg/ml, IL-4 from 0.0168 to 6.0326 pg/ml, IL-10 from 0.0112 to 4.0115 pg/ml and IFN-γfrom 0.1715 to 5.3554 pg/ml; and at 200° C. (group C2), IL-2 varied from 0.3105 to 1.0916 pg/ml, IL-4 from 0.0152 to 2.4185 pg/ml, IL-10 from 0.0098 to 20.0142 pg/ml and IFN-γfrom 0.1665 to 1.0347 pg/ml.

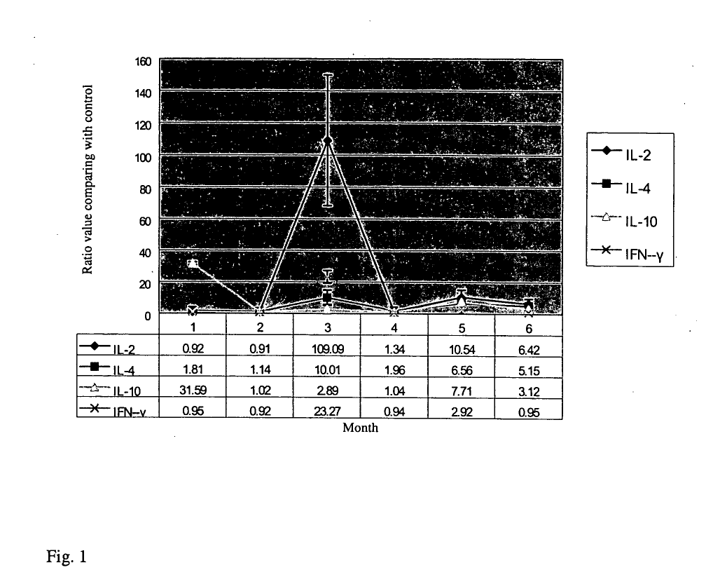

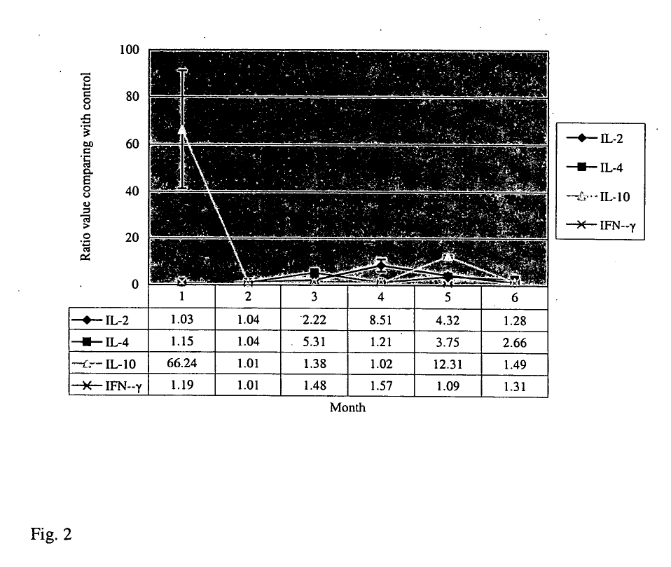

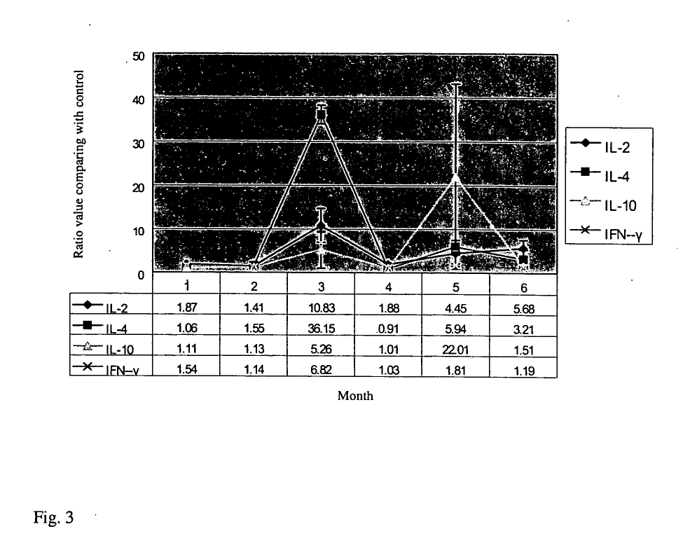

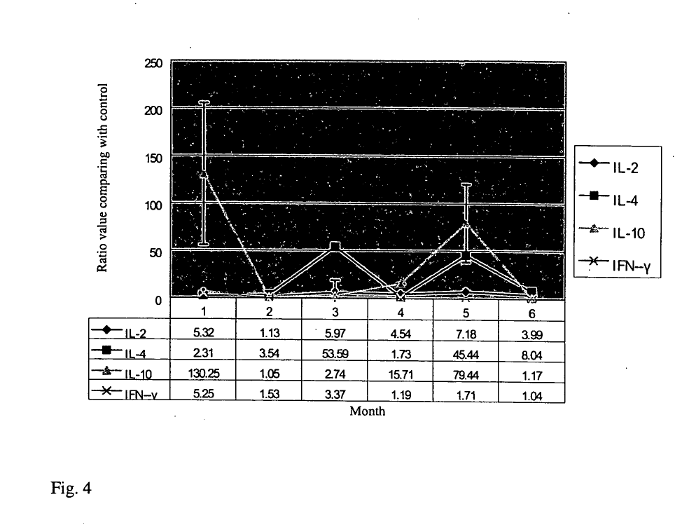

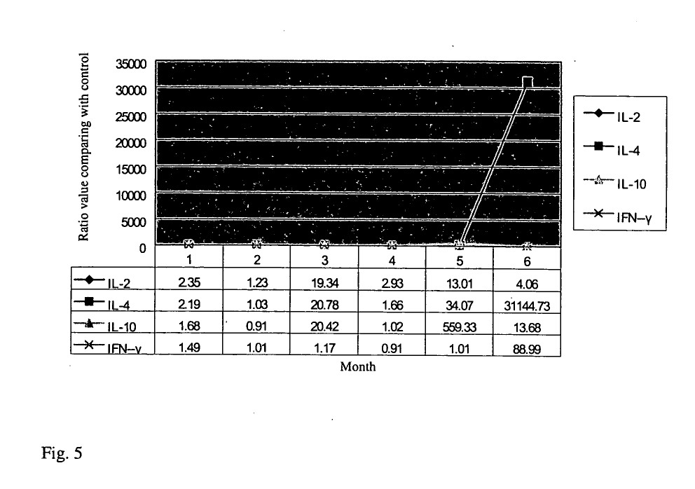

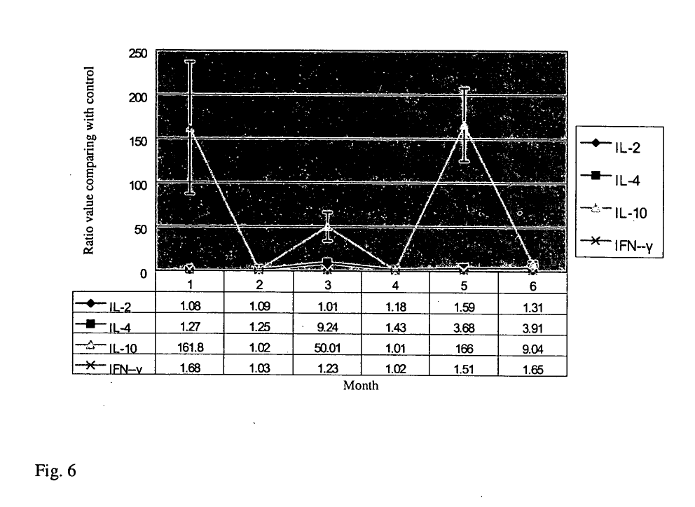

FIGS. 1-6 compare the varied cytokine ratio for mice given 50% or 75% Canavalia ensiformis seed roasted at 180° C., 190° C. and 200° C. with those in control groups (P<0.05). In group A1, serum IL-2 and IFN-γincreased significantly in month three, IL-10 increased significantly in the first month and then declined rapidly in the following months (P<0.05). IL-4 increased less markedly than IL-10 in month 3. In group A2, only serum IL-10 varied significantly and reached a maximum in the first month and a lower maximum in month five. In group B1, four tested cytokines, i.e. IL-2, IL-4, IL-10 and IFN-γ, increased in month three, and IL-10 reached a much higher maximum in month five. In group B2, cytokines IL-4 and IL-10 increased much more significantly (P<0.05) than cytokines IL-2 and IFN-γ. IL-4 increased significantly in months three and five, and IL-10 increased significantly in months one and five (P<0.05). In group C1, compared with group D, four tested cytokines, i.e. IL-2, IL-4, IL-10 and IFN-γ, have significant variation, but the concentration ratio for IL-4 increased to 31144.73 in month six, and for IL-10 increased to 559.33 in month 5. In group C2, cytokines IL-2 and IFN-γwere not significantly increased (P>0.05) but cytokines IL-4 and IL-10 increased significantly (P<0.05). IL-10 increased in months one, three and five respectively and its level was much higher than that of IL-4, which increased most significantly (P<0.05) in month three.

Discussion

Studies were performed to investigate the effect of processing on the nutritive value of Canavalia ensiformis or the activity of antinutritional and toxic factors in Balb/C mice. Bressani and Sosa (1990) reported that pressure-cooking alone and roasting at 170 □for 15 min destroyed the antiphysiological factors in Canavalia but possibly also damaged its protein quality. Melo and D'Souza (2000) mixed Canavalia ensiformis seed meal extract with crude cell-free extract to precipitate all the invertase. Babar et al. (Babar et al., 1988) found that treating the seeds with soaking for 24 hours followed by cooking for 20 min not only reduced the polyphenol content, but also had equally effective in destroying the TI activity. Risso and Montilla (1992) showed that the autoclaved (121° C./15 psi/90 min) Canavalia flour substantially improved porcine growth, but it was not effective to eliminate nor to minimize the toxic effects of the raw Canavalia. Although animals may reject food containing Con A (Larue-Achagiotis et al., 1992; Vargas et al., 1996) the mice in this study demonstrated no obvious rejection of food, suggesting that the amount of Con A in the six different diets is tolerable for the Balb/C mouse. This is consistent with the results reported by Mendez et al. (1998), who have demonstrated that broiler chicks can tolerate a daily intake of 100 mg of Con A, a constituent of Canavalia ensiformis seeds, over a six week period without growth being affected. The toxicity of the jack bean seed was also noted. Since neither significant death rate nor pathological change in the mice was observed in this study, it is unlikely that the jack bean seed treated by the indicated process was toxic to the mice at the set percentage of the meal.

Mendez et al. (1998) has studied the immune response stimulated by the jack bean seed. They (1998) found that Con A binds to the cells of the gastrointestinal tract, is absorbed into the general circulation and, eventually, elicits an immunological response without affecting the production of antibodies to Brucella abortus. Our in vivo studies also indicated that Canavalia ensiformis can activate T-cell immune response by stimulating the secretion of TH1/TH2—pattern cytokines (Tsai et al., 2000). The CD4+ lymphocytes can be grouped as TH1and TH2. When stimulated by polyclonal mitogens like Con A, they rapidly enter the G1 phase and progress through the cell cycle. If an organism triggers the release of IL-2 and IFN-γ, the TH subset that develops will bias it towards TH1, whereas release of IL-4 and IL-10 will bias it towards TH2. Thus TH1 cells release IL-2 and IFN-γ, while TH2 cells release IL-4 and IL-10. Once the TH subset has been established, either of these patterns of response is able to promote itself while suppressing the other.

In this invention, when mice were fed with Canavalia ensiformis seeds roasted at 180□ both TH1- and TH2-pattern cytokines were varied significantly (P<0.05). In group A1, serum TH1-pattern cytokines, IL-2 and IFN-γ, increased significantly in month three; IL-10 increased significantly in the first month and declined rapidly in month two, and IL-4 increased less markedly than IL-2 and IFN-γin month 3. In group A2, only serum IL-10 varied significantly and reached a higher maximum in the first month and the lower maximum in month five. When fed with seeds roasted at 190□; In group B1, both TH1- (IL-2 and IFN-γ) and TH2-pattern (IL-4 and IL-10) cytokines in month three were significantly (P<0.05) increased, and IL-10 in month five reached a much higher maximum. In group B2, TH2-pattern cytokines increased much more significantly than TH1-pattern cytokines. IL-4 increased significantly in months three and five, and IL-10 increased significantly in months one and five (P<0.05). When Canavalia ensiformis seeds were roasted at 2000; In group C1, compared with group D, both TH1 and TH2-pattern cytokines have significant variation in months three and six (P<0.05), especially the concentration ratio for IL-4 increased to 31144.73 in month six. In group C2, only TH2-pattern cytokines increased significantly. IL-10 increased in months one, three and five respectively and its level was much higher than that of IL-4, which increased in month three.

These data reveal that increased secretion for both TH1 and TH2-pattern cytokines in mice serum can be achieved with a diet of 50% Canavalia ensiformis seeds roasted at 180□ for 25 min (group A1), and TH2-pattern cytokines increased earlier and more than TH1 pattern cytokines, but when Canavalia ensiformis seeds were increased to 75% in the diet, only TH2-pattern cytokine-IL10 increased rapidly with significance (P<0.05). When Canavalia ensiformis seeds were roasted at 190° C. for 15 min, only TH2-pattern cytokines increased significantly (P<0.05) in mice serum especially when mice fed with the diet with 75% Canavalia ensiformis seeds. A similar effect was identified for each cytokine when the mice fed by Canavalia ensiformis seeds that were roasted at 200° C. for 5 min, but the TH2-pattern cytokines in mice serum increased significantly in the 50% group (P<0.05), and TH1-pattern cytokines had a lower up-regulation in this group. The most obvious increased secretion of TH2-pattern cytokines in mice serum were demonstrated in group C1.

Comparing these results with other published reports (Bressani and Sosa, 1990; Babar et al., 1988; Risso and Montilla, 1992), the results suggest that dry heat can be the choice for treating Canavalia ensiformis seeds without damaging the cytokine modulation effects and is non-toxic to the mice. Different ways of treatment may have different effects on Canavalia ensiformis seed toxicity and TH1/TH2 balance. In the present invention, in vivo modulation effect of TH2-pattern cytokines (IL-4 and IL-10) were more obvious increased than TH1-pattern cytokines (IL-2 and IFN-γ) with higher heat treatment (200° C.). Given the above, it concluded that the effect of Canavalia ensiformis seed on cytokine modulation in vivo is related to its dosage and the process of heat treatment.

1. A method of preparing dry-heating seeds of Canavalia ensiformis comprising roasting the seeds of Canavalia ensiformis at more than 170-250° C. for 0.5-150 minutes.

2. The method according to claim 1 wherein the seeds are roasted at more than 170-220° C. for 1.5-100 minutes.

3. The method according to claim 1 wherein the seeds are roasted at 180-200° C. for 5-50 minutes.

4. The method according to claim 3 wherein the seeds are roasted at 180° C. for 25 minutes.

5. The method according to claim 3 wherein the seeds are roasted at 190° C. for 15 minutes.

6. The method according to claim 3 wherein the seeds are roasted at 200° C. for 5 minutes.

7. A composition for use in immunomodulation comprising dry-heating seeds of Canavalia ensiformis wherein the seeds are roasted at more than 170-250° C. for 0.5-60 minutes.

8. The composition according to claim 7, wherein the concentration of the seeds ranges from 1 to 99 weight percent.

9. The composition according to claim 8, wherein the concentration of the seeds ranges from 10 to 90 weight percent.

10. The composition according to claim 9, wherein the concentration of the seeds ranges from 30 to 80 weight percent.

11. The composition according to claim 10, wherein the concentration of the seeds ranges from 40 to 75 weight percent.

12. The composition according to claim 11, wherein the concentration of the seeds ranges from 50 to 75 weight percent.

13. A method for the treatment of diseases caused or affected by malfunction of the immune system comprises administering the concentration of 1 to 99 weight percent of dry-heating seeds of Canavalia ensiformis wherein the seeds are roasted at more than 170-250° C. for 0.5-150 minutes, to a human or animal in need of such treatment.

14. The method according to claim 13, wherein the immune diseases are infections by exogenous pathogens

15. The method according to claim 14, wherein the exogenous pathogens are viruses, bacteria, parasites or fungi.

16. The method according to claim 13, wherein the immune diseases are autoimmune diseases.

17. The method according to claim 16, wherein the autoimmune diseases are systemic lupus erythematosus, rheumatoid arthritis, type I diabetes, inflammatory bowel disease, biliary cirrhosis, uveitis, multiple sclerosis, Crohn's disease, ulcerative colitis, bullous pemphigoid, sarcoidosis, psoriasis, ichthyosis or Graves' orbitopathy.

18. The method according to claim 13, which can be applied for cancer immunotherapy.

19. The method according to claim 13, wherein the concentration of dry-heating seeds of Canavalia ensiformis is 50 to 75 weight percent.

20. The method according to claim 13, wherein the seeds are roasted at 180-200° C. for 5-100 minutes.
